(19) United States
(12) Patent Application Publication
(10) Pub. No.: US 2015/0258267 A1
(43) Pub. Date: Sep. 17, 2015

(54) PLASMA SEPARATION USING A DROP OF BLOOD

(71) Applicant: KONINKLIJKE PHILIPS N.V., EINDHOVEN (NL)

(72) Inventors: Anil Shivram Raiker, Goa (IN); Ratheesh Narayanan, Pondicherry (IN); Shrutin Ulman, Bangalore (IN)

(21) Appl. No.: 14/417,966

(22) PCT Filed: Aug. 8, 2013
(86) PCT No.: PCT/IB2013/056486

§ 371 Date: Jan. 28, 2015

(30) Foreign Application Priority Data
Aug. 8, 2012 (IN) 3257/CHE/2012

(51) Int. Cl. A61M 1/34

(52) U.S. Cl. CPC A61M 1/34

(57) ABSTRACT

Systems, combinations, and/or methods for separating plasma from a drop of blood use a capillary tube and a filter membrane having a rough side and a smooth side. The pores on the rough side of the filter membrane are large enough for blood cells to enter and be captured. The pores on the smooth side of the filter membrane are small enough to block blood cells, but allow the plasma within the drop of blood to pass through the smooth side of the filter membrane.

The present disclosure pertains to systems, combinations, and/or methods for separating plasma from merely a drop of blood, and, in particular, for using capillarity to transfer small quantities of blood and/or the plasma therein.

It is well known to extract plasma from blood by separating blood cells, for example through centrifugal force. It is known that different components of blood have different densities. It is known that different components of blood, in particular blood cells, have different particle sizes. It is known that suitably configured filters may capture, block, and/or pass particular components within blood.

Accordingly, it is an object of one or more embodiments of the present invention to provide a combination for separating plasma from blood. The combination comprises a capillary tube having a diameter ranging between 0.1 mm and 2.5 mm, and further having an interior, a first opening, and a second opening, and a filter membrane having a rough side and a smooth side, wherein the rough side includes pores large enough to capture blood cells, wherein the smooth side includes pores small enough to block blood cells. Using the combination allows separation of plasma from a quantity of blood of about 0.03 ml or less, responsive to the filter membrane being disposed in proximity of the blood, through capillarity from the capillary tube acting to transfer of one or both of plasma and/or the blood.

It is yet another aspect of one or more embodiments of the present invention to provide a method for separating plasma from blood implemented using a combination including a capillary tube having an interior and a filter membrane having a rough side and a smooth side, the rough side of the filter membrane including pores large enough to capture blood cells, the smooth side of the filter membrane including pores small enough to block blood cells, the capillary tube having a first opening and a second opening. The method comprises disposing the filter membrane in proximity of a quantity of blood of about 0.03 ml or less; and transferring one or both of plasma and/or the blood into the interior of the capillary tube through capillarity from the capillary tube.

It is yet another aspect of one or more embodiments to provide a combination for separating plasma from blood. The combination comprises first means and second means. The first means is for disposing in proximity of a quantity of blood of about 0.03 ml or less, wherein the first means has a rough side and a smooth side, the rough side including pores large enough to capture blood cells, the smooth side including pores small enough to block blood cells. The second means is for transferring one or both of plasma and/or the blood through capillarity, the second means having a first opening.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

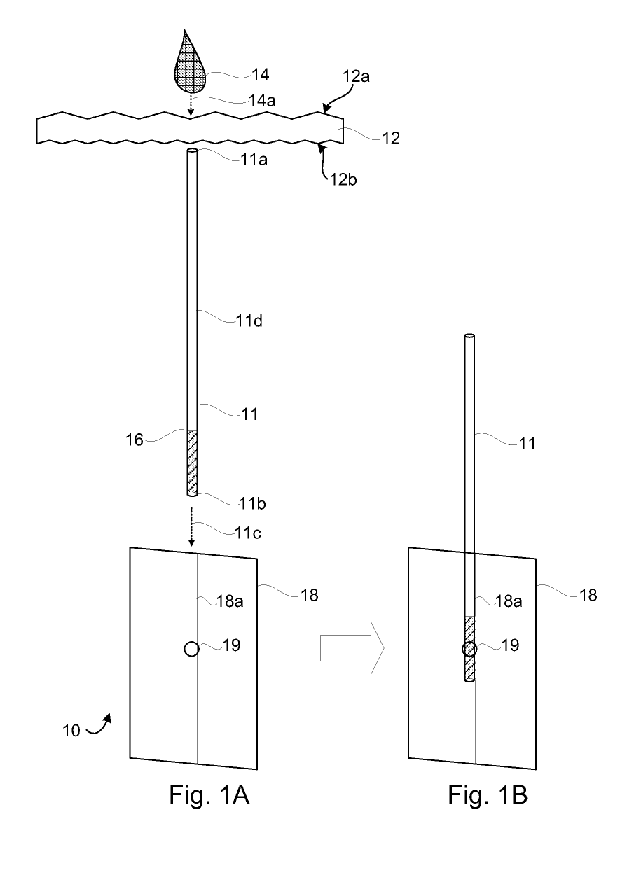

FIG. 1A-1B illustrate a system or combination for separating plasma from blood, using a capillary tube and a filter membrane in accordance with one or more embodiments;

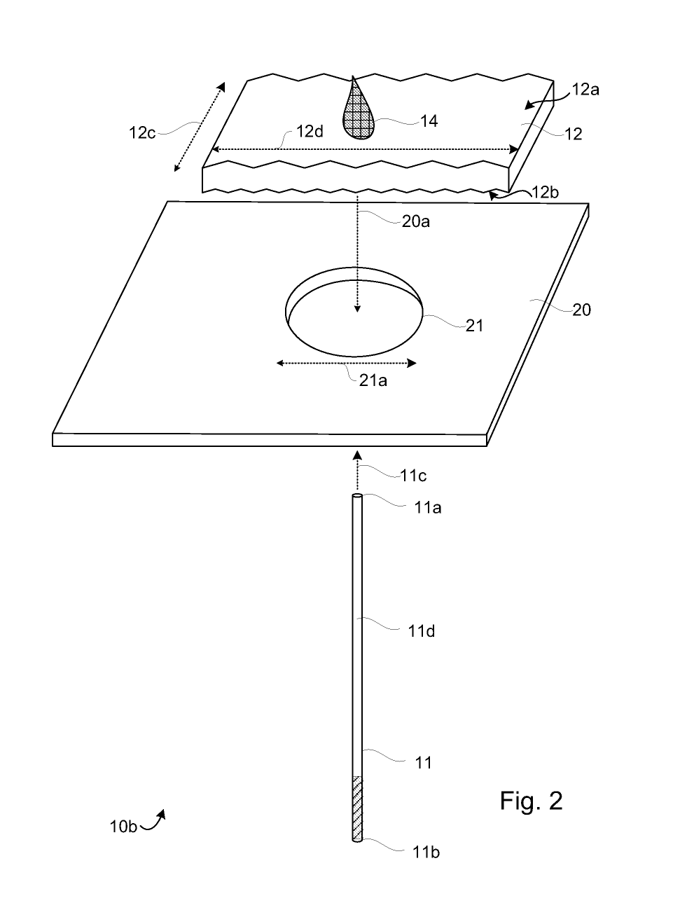

FIG. 2 illustrates a system or combination for separating plasma from blood, using a capillary tube and a filter membrane in accordance with one or more embodiments;

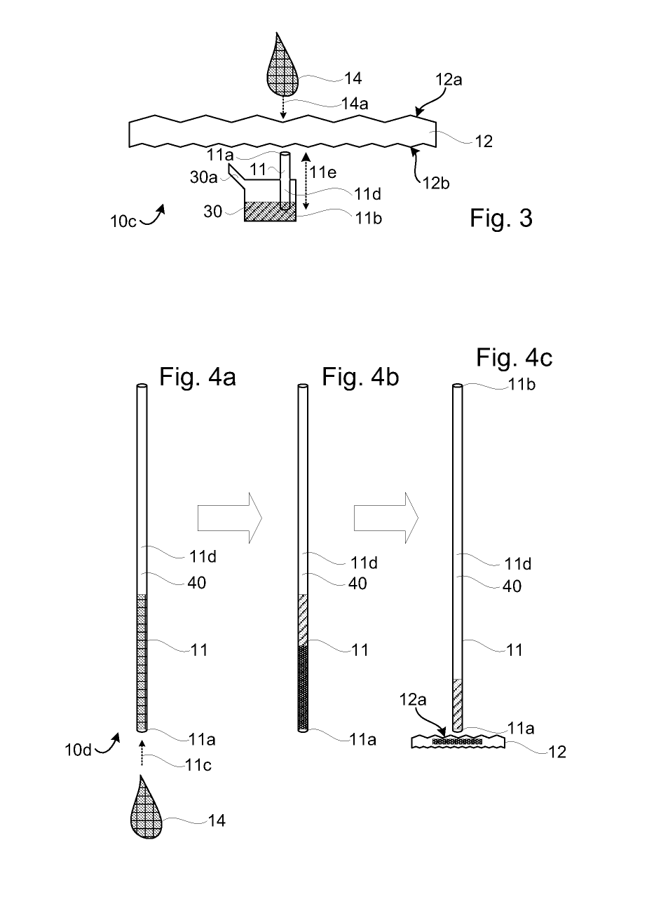

FIG. 3 illustrates a system or combination for separating plasma from blood, using a capillary tube, a filter membrane, and a well in accordance with one or more embodiments;

FIG. 4A-4B-4C illustrate a system or combination for separating plasma from blood, using a capillary tube coated with a solution in accordance with one or more embodiments;

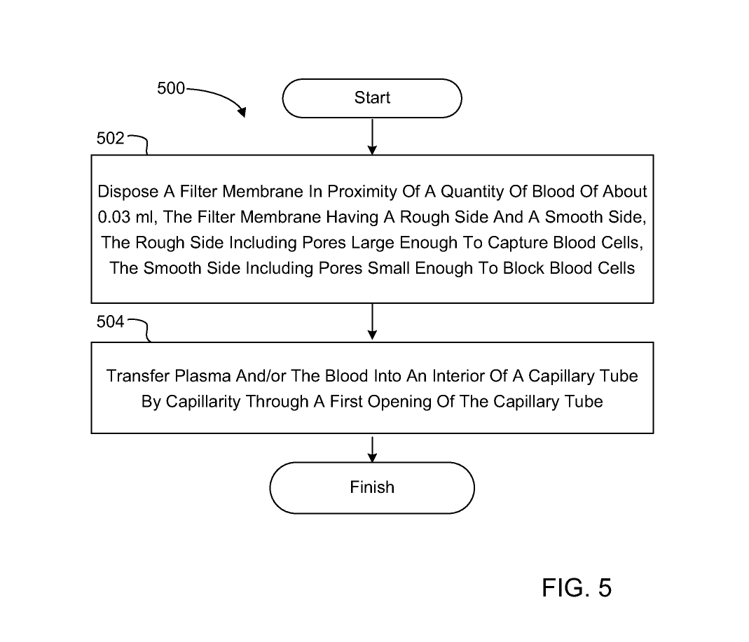

FIG. 5 illustrates a method for separating plasma from blood;

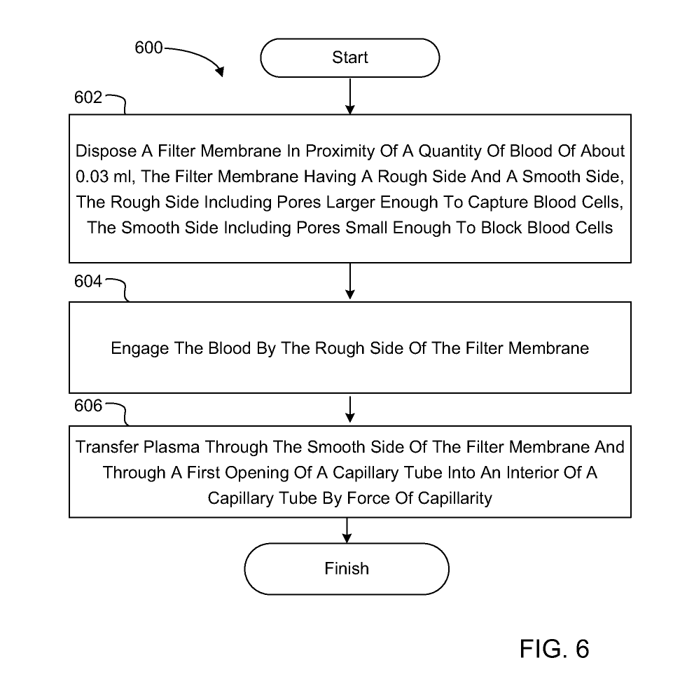

FIG. 6 illustrates a method for separating plasma from blood using a filter membrane and a capillary tube;

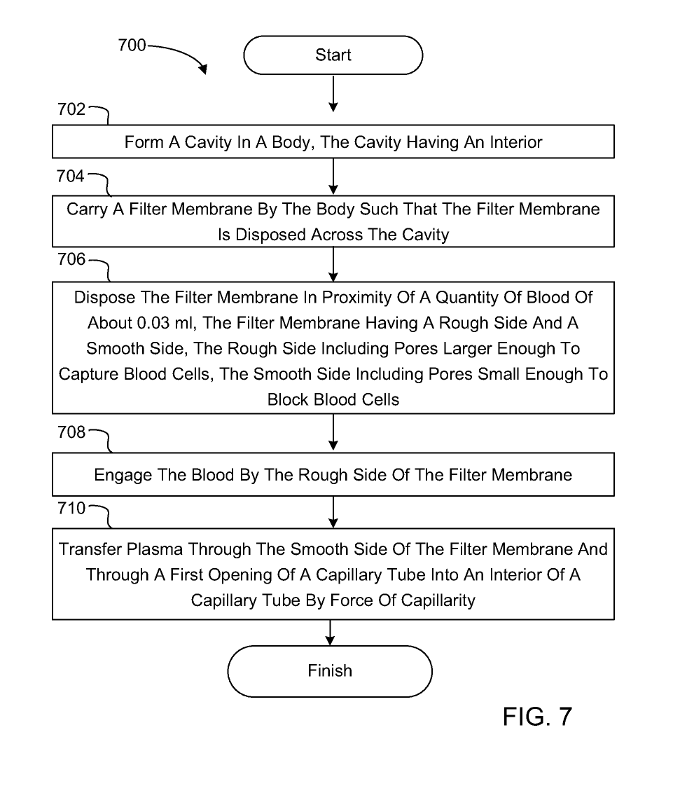

FIG. 7 illustrates a method for separating plasma from blood using a filter membrane and a capillary tube;

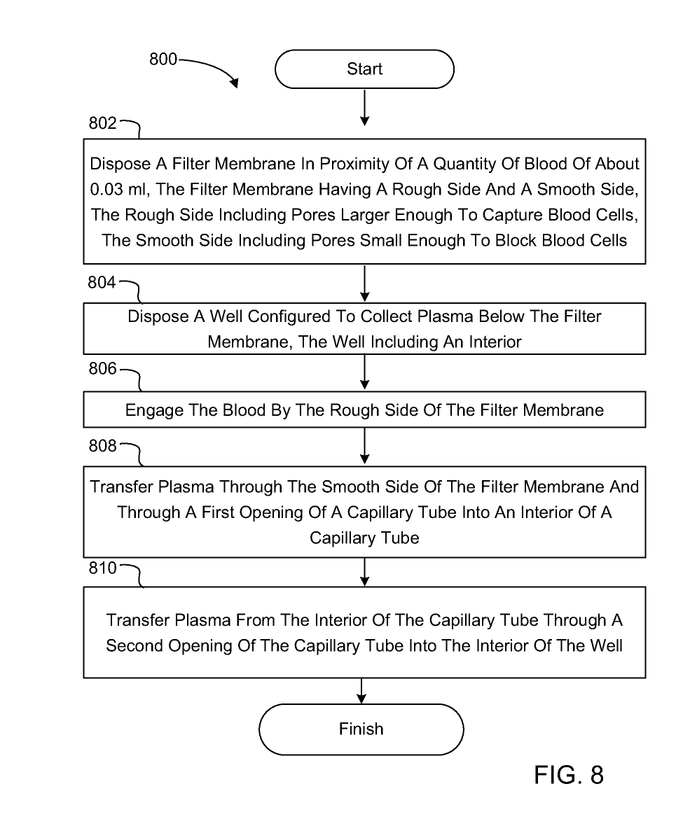

FIG. 8 illustrates a method for separating plasma from blood using a filter membrane, a capillary tube, and a well; and

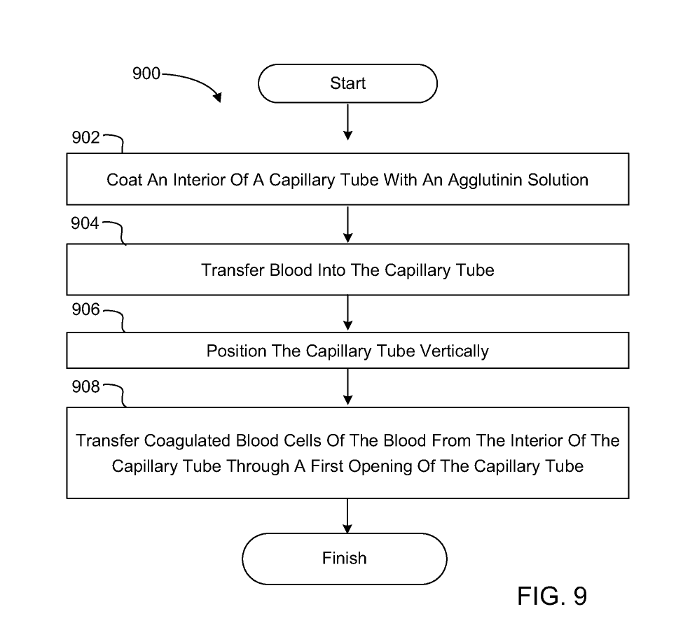

FIG. 9 illustrates a method for separating plasma from blood using a capillary tube coated in a solution.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

FIG. 1A illustrates a system 10 (or combination 10) for separating plasma from blood, using a capillary tube 11 and a filter membrane 12. The terms system 10 and combination 10 may be used interchangeably herein. Common techniques to separate plasma from blood, for example through removing blood cells from blood, require a volume of blood that is larger than a single drop of blood. The systems, combinations, and methods described herein require merely a drop of blood. The quantity of blood may be about 0.02 ml, about 0.03 ml, about 0.05 ml, between 0 ml and 0.03 ml, between 0.02 ml and 0.03 ml, between 0.03 ml and 0.05 ml, less than about 0.03 ml, less than about 0.05 ml, and/or another quantity of blood. One of various reasons to separate and/or extract plasma from blood is to enable spectrophotometric analysis. For example, for neonates and/or infants, a spectrophotometric reading of plasma may be used to characterize the occurrence and/or quantity of one or more substances therein, such as, by way of non-limiting example, bilirubin.

Common techniques for separating plasma from blood may use centrifugal force, for example applied in a clinical laboratory, to separate relatively denser components and/or substances within blood from relatively less dense components and/or substances within blood.

System 10 includes one or more of a capillary tube 11, a filter membrane 12, a cartridge 18, and/or other components. Filter membrane 12 includes two sides, which may be referred to as a rough side 12a and a smooth side 12b. Rough side 12a includes pores large enough to pass blood cells. The pores on rough side 12a may be referred to as capillary pores. The pores on rough side 12a of filter membrane 12 may draw in liquids and/or liquid substances by capillarity. Blood cells may be captured through rough side 12a. Smooth side 12b includes pores small enough to block blood cells. In other words, blood cells may be trapped within filter membrane 12 because they cannot pass through smooth side 12b of filter membrane 12. As depicted in FIG. 1A, a drop of blood 14, e.g. having a volume of about 0.03 ml or less, relatively moving in a direction indicated by directional indicator 14a, may engage rough side 12a of filter membrane 12. As depicted in FIG. 1A, filter membrane 12 is shown in a cross-sectional view. Responsive to drop of blood 14 engaging filter membrane 12, blood permeates within filter membrane 12. In some embodiments, the drop of blood may be stationary and filter membrane 12 may be disposed in proximity of the drop of blood such that the blood engages filter membrane 12, in particular rough side 12a of filter membrane 12. It may take a period of time for the drop of blood to engage filter membrane 12, for example about 10 seconds, about 15 seconds, about 20 seconds, about 25 seconds, about 30 seconds, about 40 seconds, about 50 seconds, about 60 seconds, and/or another suitable amount of time that is not prohibitively long for a caregiver to wait for separating plasma from blood, in particular during interaction with a patient.

Capillary tube 11 may have a first opening 11a, a second end or second opening 11b, and an interior 11d. The diameter of capillary tube 11 may be about 0.05 mm, about 0.1 mm, about 0.25 mm, about 0.5 mm, about 1.0 mm, about 2.5 mm, ranging between about 0.05 mm and 0.25 mm, ranging between about 0.1 mm and about 2.5 mm, between about 0.5 mm and about 1.5 mm, and/or another suitable diameter such that capillary tube 11 is capable of transferring plasma and/or blood by force of capillarity, despite or in addition to forces of gravity and/or flow resistance due to a filter membrane. Capillary tube 11 may vary in length between about 1 mm and about 100 mm. In a preferred embodiment, the length of capillary tube 11 is about 60 mm.

Responsive to capillary tube 11, particularly first opening 11a thereof, being disposed in proximity of filter membrane 12 and/or engaging filter membrane 12, particularly smooth side 12b thereof, capillary forces from capillary tube 11 may act to transfer plasma from blood through smooth side 12b from filter membrane 12 into interior 11d of capillary tube 11. The terms “capillary forces” and “capillarity” may be used interchangeably herein. Within interior 11d of capillary tube 11, plasma may pool at end 11b, through gravity, such that the amount of plasma is indicated by level 16 as depicted in FIG. 1A. In some embodiments, filter membrane 12 and capillary tube 11 may be integrated, and/or otherwise combined.

In some embodiments, capillary tube 11 may be configured to fit in a slot 18a of cartridge 18. Slot 18a may be configured to have a size, width, and/or diameter capable of receiving at least part of capillary tube 11. Responsive to fitting capillary tube 11 into slot 18a of cartridge 18, as depicted by directional indicator 11c in FIG. 1A and the combination of capillary tube 11 and cartridge 18 as depicted in FIG. 1B, cartridge 18 may be used for analysis, e.g. spectrophotometric analysis. Cartridge 18 may be an immunoassay cartridge. Cartridge 18 may include a reading window 19, through which a spectrophotometric reading may be performed. Responsive to reception of at least part of capillary tube 11 in slot 18a, such as for example depicted by FIG. 1B, at least a segment of capillary tube 11 may be aligned with reading window 19 of cartridge 18. In some embodiments, capillary tube 11 and cartridge 18 may be integrated, and/or otherwise combined.

FIG. 2 illustrates a system (or combination) 10b for separating plasma from drop of blood 14, using capillary tube 11, filter membrane 12, a slide 20, and/or other components. Capillary tube 11 and filter membrane 12 are substantially the same as or similar to the respective components of system 10 depicted in FIG. 1A. Referring to FIG. 2, filter membrane 12 is depicted in an isometric view, which illustrates a filter membrane length 12d and a filter membrane width 12c. The rectangular shape of filter membrane 12 as depicted is exemplary, and not intended to be limiting in any way. For example, the shape of filter membrane 12 may be circular, oval, polygonal, and/or other suitable shapes. In some embodiments, filter membrane 12 may be circular having a diameter (as well as filter membrane length 12d and filter membrane width 12c) of about 3 mm, about 6 mm, about 7.5 mm, about 10 mm, about 15 mm, and/or another diameter.

Slide 20 of system 10b in FIG. 2 may include a body in which a cavity 21 is formed. Slide 20 may be a substantially rigid material, such as glass, ceramic, plastic, and/or another suitable rigid material such that filter membrane 12 may be carried and/or held across cavity 21 and/or an interior of cavity 21. Filter membrane 12 may be disposed in proximity to cavity 21 by relatively moving filter membrane 12 in a direction indicated by directional indicator 20a such that at least part of filter membrane 12 engages slide 20. The circular shape of cavity 21 as depicted is exemplary, and not intended to be limiting in any way. For example, the shape of cavity 21 may be circular, oval, polygonal, and/or other suitable shapes. In some embodiments, cavity 21 may be circular having a cavity diameter 21a of about 3 mm, about 5 mm, about 7 mm, about 10 mm, about 15 mm, and/or another diameter. In some embodiments, the size of cavity 21 and the size of filter membrane may be selected such that filter membrane 12 can cover all or most of cavity 21, for example by fitting across cavity 21.

Responsive to filter membrane 12 engaging slide 20, filter membrane may be fixed in place by glue, adhesive, a clamp, tape, and/or any other mechanism that reduces relative motion of filter membrane 12 relative to slide 20 and/or cavity 21, temporarily or permanently. In some embodiments, a piece of tape may be used that fully covers the length and width of filter membrane 12 except for a hole in the center through which drop of blood 14 can engage filter membrane 12. The hole may be circular, and may have a diameter of about 2 mm, about 5 mm, about 6 mm, about 8 mm, about 10 mm, and/or another diameter. In some embodiments, the size of the hole in the tape and the size of filter membrane may be selected such that filter membrane 12 can cover all or most of the hole.

Responsive to drop of blood 14 engaging filter membrane 12, blood permeates within filter membrane 12. In some embodiments, the drop of blood may be stationary and filter membrane 12 may be disposed in proximity of the drop of blood such that the blood engages filter membrane 12, in particular rough side 12a of filter membrane 12. It may take a period of time for the drop of blood to engage filter membrane 12, for example about 10 seconds, about 15 seconds, about 20 seconds, about 25 seconds, about 30 seconds, about 40 seconds, about 50 seconds, about 60 seconds, and/or another suitable amount of time that is not prohibitively long for a caregiver to wait for separating plasma from blood.

Responsive to capillary tube 11, particularly first opening 11a thereof, being disposed in proximity of filter membrane 12 and/or engaging filter membrane 12, particularly smooth side 12b thereof, capillary forces from capillary tube 11 may act to transfer plasma from blood through smooth side 12b from filter membrane 12 into interior 11d of capillary tube 11. Within interior 11d of capillary tube 11, plasma may pool at end 11b, through gravity. In some embodiments, filter membrane 12 and capillary tube 11 may be integrated, and/or otherwise combined. Once plasma is collected within capillary tube 11, it may be analyzed through one or more techniques, including but not limited to spectrophotometric analysis.

FIG. 3 illustrates a system (or combination) 10c for separating plasma from drop of blood 14, using capillary tube 11 having first opening 11a and second opening 11b, filter membrane 12, a well 30, and/or other components. Capillary tube 11 and filter membrane 12 are substantially the same as or similar to the respective components of system 10 depicted in FIG. 1A, though capillary tube may be shorter. Referring to FIG. 3 and system 10c, capillary tube length 11e of capillary tube may be about 1 mm, about 2 mm, at least about 2 mm, about 5 mm, about 10 mm, less than about 10 mm, about 20 mm, and/or another suitable length to transfer plasma from filter membrane 12 into well 30. In some embodiments, capillary tube 11 may include a predetermined angle between first opening 11a and second opening 11b. The predetermined angle may range between about 0 degrees and about 90 degrees, less than about 90 degrees, less than about 60 degrees, and/or another suitable angle to transfer plasma from filter membrane 12 to well 30.

Well 30 of system 10c is configured to collect plasma within its interior. The shape and volume of well 30 are not limited by the exemplary embodiment depicted in FIG. 3. Well 30 may be disposed below filter membrane 12. In some embodiments at least part of well 30 may be substantially or nearly level with at least part of filter membrane 12 such that the transfer of plasma through smooth side 12b of filter membrane 12 into the interior of well 30 occurs through an embodiment of capillary tube 11 that includes the predetermined angle discussed above. Well 30 may include an outlet 30a through which air may leave the interior of well 30 responsive to plasma being transferred into the interior of well 30.

Responsive to drop of blood 14 engaging filter membrane 12, e.g. by the blood moving as indicated by directional indicator 14a, blood permeates within filter membrane 12. In some embodiments, the drop of blood may be stationary and filter membrane 12 may be disposed in proximity of the drop of blood such that the blood engages filter membrane 12, in particular rough side 12a of filter membrane 12. It may take a period of time for the drop of blood to engage filter membrane 12, for example about 10 seconds, about 15 seconds, about 20 seconds, about 25 seconds, about 30 seconds, about 40 seconds, about 50 seconds, about 60 seconds, and/or another suitable amount of time that is not prohibitively long for a caregiver to wait for separating plasma from blood, in particular during patient interaction.

Responsive to capillary tube 11, particularly first opening 11a thereof, being disposed in proximity of filter membrane 12 and/or engaging filter membrane 12, particularly smooth side 12b thereof, capillary forces from capillary tube 11 may act to transfer plasma from blood through smooth side 12b from filter membrane 12 into interior 11d of capillary tube 11. Once the plasma is disposed and/or transferred into interior 11d of capillary tube 11, gravity, capillarity, and/or other forces may act to transfer the plasma into well 30 through second opening 11b, which may be held in a position such that second opening 11b is disposed downwardly. In some embodiments, filter membrane 12, capillary tube 11, and/or well 30 may be integrated, and/or otherwise combined. Once plasma is collected within well 30, it may be analyzed through one or more techniques, including but not limited to spectrophotometric analysis.

FIG. 4A-4B-4C illustrate a system (or combination) 10d for separating plasma from drop of blood 14, using capillary tube 11 having one or more of first opening 11a and second opening 11b, filter membrane 12, an agglutinin solution 40, and/or other components. Capillary tube 11 is substantially the same as or similar to capillary tube 11 of system 10 depicted in FIG. 1A. Referring to FIG. 4A, capillary tube 11 is coated with agglutinin solution 40. In particular, interior 11d of capillary tube is coated with agglutinin solution 40.

Responsive to drop of blood 14 engaging capillary tube 11, e.g. by the blood moving as indicated by directional indicator 11c in FIG. 4A, blood is transferred by capillarity through first opening 11a into interior 11d of capillary tube 11. In some embodiments, the drop of blood may be stationary and capillary tube 11 may be disposed in proximity of the drop of blood such that the blood engages capillary tube 11. It may take a period of time for the drop of blood to engage and/or mix with agglutinin solution 40, for example about 10 seconds, about 15 seconds, about 20 seconds, about 25 seconds, about 30 seconds, between about 20 seconds and about 30 seconds, about 40 seconds, about 50 seconds, about 60 seconds, and/or another suitable amount of time that is not prohibitively long for a caregiver to wait for separating plasma from blood. The agglutinin solution causes the blood to coagulate such that certain particles form a thickened mass. Responsive to the period of time passing, capillary tube 11 is disposed and/or positioned vertically, such that the relatively denser components and/or substances, such as the thickened mass, in drop of blood 14 separate from relatively less dense components and/or substances within the blood, such as plasma. In other words the relatively denser components and/or substances within interior 11d sink towards first opening 11a as depicted in FIG. 4B by force of gravity. Note that gravity may overcome capillarity from capillary tube 11 for the thickened mass.

As illustrated in FIG. 4C, the relatively denser components and/or substances at or near first opening 11a may be transferred out of capillary tube 11, for example into filter membrane 12 and/or another suitable receptacle. In other words, interior 11d may be drained, e.g. into filter membrane 12 through a combination of gravity and capillary forces from the capillary pores on rough side 12a of filter membrane 12, such that plasma remains within interior 11d. In some implementations, gas and/or air pressure applied through second opening 11b of capillary tube may be used to force the relatively denser components and/or substances at or near first opening 11a to be transferred out of capillary tube 11. Once plasma is collected and/or remaining within capillary tube 11, it may be analyzed through one or more techniques, including but not limited to spectrophotometric analysis.

FIGS. 5-9 illustrate methods 500-900 for separating plasma from blood. The operations of methods 500-900 presented below are intended to be illustrative. In certain embodiments, methods 500-900 may be accomplished with one or more additional operations not described, and/or without one or more of the operations discussed. Additionally, the order in which the operations of methods 500-900 are illustrated in FIGS. 5-9 and described below is not intended to be limiting.

In certain embodiments, methods 500-900 may be implemented using one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of methods 500-900 in response to instructions stored electronically on an electronic storage medium. The one or more processing devices may include one or more devices configured through hardware, firmware, and/or software to be specifically designed for execution of one or more of the operations of methods 500-900.

Referring to FIG. 5, at an operation 502, a filter membrane is disposed in proximity of a predetermined quantity of blood. The filter membrane has a rough side and a smooth side. The rough side includes pores large enough to capture blood cells. The smooth side includes pores small enough to block blood cells. In some embodiments, operation 502 is performed by a filter membrane the same as or similar to filter membrane 12 (shown in FIG. 1A and described herein).

At an operation 504, plasma and/or blood is transferred into an interior of a capillary tube by capillarity through a first opening of the capillary tube. In some embodiments, operation 504 is performed by a capillary tube the same as or similar to capillary tube 11 (shown in FIG. 1A and described herein).

Referring to FIG. 6, at an operation 602, a filter membrane is disposed in proximity of a predetermined quantity of blood. The filter membrane has a rough side and a smooth side. The rough side includes pores large enough to capture blood cells. The smooth side includes pores small enough to block blood cells. In some embodiments, operation 602 is performed by a filter membrane the same as or similar to filter membrane 12 (shown in FIG. 1A and described herein).

At an operation 604, the blood is engaged by the rough side of the filter membrane. In some embodiments, operation 604 is performed by a rough side of a filter membrane the same as or similar to rough side 12a of filter membrane 12 (shown in FIG. 1A and described herein).

At an operation 606, plasma is transferred through the smooth side of the filter membrane and through a first opening in the capillary tube into an interior of a capillary tube by force of capillarity. In some embodiments, operation 606 is performed by a capillary tube the same as or similar to capillary tube 11 (shown in FIG. 1A and described herein).

Referring to FIG. 7, at an operation 702, a cavity is formed in a body, the cavity having an interior. In some embodiments, operation 702 is performed by a body the same as or similar to slide 20 (shown in FIG. 2 and described herein).

At an operation 704, a filter membrane is carried by the body such that the filter membrane is disposed across the cavity. In some embodiments, operation 704 is performed by a body the same as or similar to slide 20 (shown in FIG. 2 and described herein).

At an operation 706, the filter membrane is disposed in proximity of a predetermined quantity of blood. The filter membrane has a rough side and a smooth side. The rough side includes pores large enough to capture blood cells. The smooth side includes pores small enough to block blood cells. In some embodiments, operation 706 is performed by a filter membrane the same as or similar to filter membrane 12 (shown in FIG. 2 and described herein).

At an operation 708, the blood is engaged by the rough side of the filter membrane. In some embodiments, operation 708 is performed by a rough side of a filter membrane the same as or similar to rough side 12a of filter membrane 12 (shown in FIG. 2 and described herein).

At an operation 710, plasma is transferred through the smooth side of the filter membrane and through a first opening in the capillary tube into an interior of a capillary tube by force of capillarity. In some embodiments, operation 710 is performed by a capillary tube the same as or similar to capillary tube 11 (shown in FIG. 2 and described herein).

Referring to FIG. 8, at an operation 802, a filter membrane is disposed in proximity of a predetermined quantity of blood. The filter membrane has a rough side and a smooth side. The rough side includes pores large enough to capture blood cells. The smooth side includes pores small enough to block blood cells. In some embodiments, operation 802 is performed by a filter membrane the same as or similar to filter membrane 12 (shown in FIG. 3 and described herein).

At an operation 804, a well configured to collect plasma is disposed below the filter membrane. The well includes an interior. In some embodiments, operation 804 is performed by a well the same as or similar to well 30 (shown in FIG. 3 and described herein).

At an operation 806, the blood is engaged by the rough side of the filter membrane. In some embodiments, operation 806 is performed by a rough side of a filter membrane the same as or similar to rough side 12a of filter membrane 12 (shown in FIG. 3 and described herein).

At an operation 808, plasma is transferred through the smooth side of the filter membrane and through a first opening in the capillary tube into an interior of a capillary tube by force of capillarity. In some embodiments, operation 808 is performed by a capillary tube the same as or similar to capillary tube 11 (shown in FIG. 3 and described herein).

At an operation 810, plasma is transferred from the interior of the capillary tube through a second opening in the capillary tube into the interior of the well by force of gravity. In some embodiments, operation 810 is performed by a capillary tube and well the same as or similar to capillary tube 11 and well 30 (shown in FIG. 3 and described herein).

Referring to FIG. 9, at an operation 902, an interior of a capillary tube is coated with an agglutinin solution. In some embodiments, operation 902 is performed by an agglutinin solution the same as or similar to agglutinin solution 40 (shown in FIG. 4A and described herein).

At an operation 904, blood is transferred through a first opening of a capillary tube into the interior of a capillary tube by force of capillarity. In some embodiments, operation 904 is performed by a capillary tube the same as or similar to capillary tube 11 (shown in FIG. 4A and described herein).

At an operation 906, responsive to a period of time passing, the capillary tube is positioned vertically. The period of time is for the blood in the interior of the capillary tube to mix with the agglutinin solution. In some embodiments, operation 906 is performed by a capillary tube the same as or similar to capillary tube 11 (shown in FIGS. 4A-4B and described herein).

At an operation 908, blood cells are transferred from the interior of the capillary tube through, e.g., the first opening in the capillary tube by force of gravity and/or by force of capillarity from the pores in the rough side of the filter membrane. In doing so, plasma remains within capillary tube 11. In some embodiments, operation 908 is performed by a first opening of a capillary tube and by the rough side of the filter membrane the same as or similar to first opening 11a of capillary tube 11 and rough side 12a of filter membrane 12 (shown in FIG. 4C and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the embodiment has been described in detail for the purpose of illustration based on what is currently considered to be the most practical and preferred embodiments, it is to be understood that such detail is solely for that purpose and that the embodiment is not limited to the disclosed embodiments, but, on the contrary, is intended to cover modifications and equivalent arrangements that are within the spirit and scope of the appended claims. For example, it is to be understood that the present embodiment contemplates that, to the extent possible, one or more features of any embodiment can be combined with one or more features of any other embodiment.

1. A combination for separating plasma from blood, the combination including:
a capillary tube having a diameter ranging between 0.1 mm and 2.5 mm, and further having an interior, a first opening, and a second opening; and
a filter membrane having a rough side and a smooth side, wherein the rough side includes pores large enough to capture blood cells, wherein the smooth side includes pores small enough to block blood cells,
wherein using the combination of the capillary tube and the filter membrane together allows separation of plasma from a quantity of blood of 0.03 ml or less, responsive to the filter membrane being disposed in proximity of the blood such that the blood enters the filter membrane through the rough side of the filter membrane, by virtue of capillarity from the capillary tube acting to transfer the plasma through the smooth side of the filter membrane.

2. The combination of claim 1, wherein, responsive to the blood engaging the rough side of the filter membrane, the first opening of the capillary tube is disposed at or near the smooth side of the filter membrane such that the filter membrane is disposed between the capillary tube and the blood, wherein capillarity from the capillary tube acts to transfer plasma from the blood through the first opening of the capillary tube into the interior of the capillary tube.

3. The combination of claim 1, further comprising:
a cavity formed in a body configured to carry the filter membrane, the cavity having an interior, wherein the filter membrane is disposed across the cavity and between the interior of the cavity and the blood such that, responsive to the blood engaging the rough side of the filter membrane, responsive to a period of time for the blood to engage the filter membrane passing, and responsive to the first opening of the capillary tube being disposed at or near the smooth side of the filter membrane, capillarity from the capillary tube acts to transfer plasma through the smooth side of the filter membrane and through the first opening of the capillary tube into the interior of the capillary tube.

4. The combination of claim 1, wherein the capillary tube includes a predetermined angle between the first opening and the second opening of the capillary tube, the capillary tube having a length of at least 2 mm, the combination further comprising:
a well configured to collect plasma, the well having an interior, wherein the well is disposed below the filter membrane,
wherein, responsive to the blood engaging the rough side of the filter membrane, the first opening of the capillary tube is disposed at or near the smooth side of the filter membrane such that the filter membrane is disposed between the first opening of the capillary tube and the blood, wherein the second opening of the capillary tube is disposed at or near the well such that capillarity from the capillary tube acts to transfer plasma through the smooth side of the filter membrane and through the first opening of the capillary tube into the interior of the capillary tube, and wherein one or both of gravity and/or capillarity act to transfer plasma from the interior of the capillary tube through the second opening of the capillary tube into the interior of the well.

5. The combination of claim 1, further comprising:
an agglutinin solution that coats the interior of the capillary tube, wherein, responsive to capillarity from the capillary tube acting to transfer the blood into the interior of the capillary tube and responsive to a period of time for the blood in the interior of the capillary tube to mix with the agglutinin solution passing, the capillary tube is positioned vertically, wherein the first opening of the capillary tube is disposed in proximity of the rough side of the filter membrane such that gravity acts to transfer blood cells from the interior of the capillary tube through the first opening of the capillary tube to engage the rough side of the filter membrane.

6. A method for separating plasma from blood implemented using a combination including a capillary tube having an interior and a filter membrane having a rough side and a smooth side, the rough side of the filter membrane including pores large enough to capture blood cells, the smooth side of the filter membrane including pores small enough to block blood cells, the capillary tube having a first opening and a second opening, the method comprising;
disposing the filter membrane in proximity of a quantity of blood of 0.03 ml or less such that the blood enters the filter membrane through the rough side of the filter membrane; and
transferring the plasma through the smooth side of the filter membrane and into the interior of the capillary tube by virtue of capillarity from the capillary tube.

7. The method of claim 6, further comprising:
disposing the first opening of the capillary tube at or near the smooth side of the filter membrane such that the filter membrane is disposed between the capillary tube and the blood,
wherein transferring includes transferring, by capillarity from the capillary tube, plasma through the first opening of the capillary tube into the interior of the capillary tube.

8. The method of claim 6, further comprising:
forming a cavity in a body, the cavity having an interior;
carrying the filter membrane by the body;
disposing the filter membrane across the cavity and between the interior of the cavity and the blood;
engaging the blood by the rough side of the filter membrane; and
responsive to a period of time for the blood to engage the rough side of the filter membrane passing, and responsive to the first opening of the capillary tube being disposed at or near the smooth side of the filter membrane, transferring, by capillarity, plasma through the smooth side of the filter membrane and through the first opening of the capillary tube into the interior of the capillary tube.

9. The method of claim 6, further comprising:
disposing a well configured to collect plasma below the filter membrane, wherein the well includes an interior;
engaging the blood by the rough side of the filter membrane;
responsive to a period of time for the blood to engage the rough side of filter membrane passing, and responsive to the first opening of the capillary tube being disposed at or near the smooth side of the filter membrane such that the filter membrane is disposed between the first end of the capillary tube and the blood, transferring plasma through the smooth side of the filter membrane and through the first opening of the capillary tube into the interior of the capillary tube by one or both of capillarity and/or gravity; and
transferring plasma from the interior of the capillary tube through the second opening of the capillary tube into the interior of the well by one or both of capillarity and/or gravity.

10. The method of claim 6, further comprising:
coating the interior of the capillary tube with an agglutinin solution;
transferring, by capillarity, the blood into the interior of the capillary tube;
responsive to a period of time for the blood in the interior of the capillary tube to mix with the agglutinin solution passing, positioning the capillary tube vertically;
disposing the first opening of the capillary tube in proximity of the rough side of the filter membrane; and
transferring, by gravity and capillarity from the rough side of the filter membrane, blood cells from the interior of the capillary tube through the first opening of the capillary tube to engage the rough side of the filter membrane.

11. A combination for separating plasma from blood, the combination comprising:
first means for disposing in proximity of a quantity of blood of 0.03 ml or less, wherein the first means has a rough side and a smooth side, the rough side including pores large enough to capture blood cells, the smooth side including pores small enough to block blood cells, wherein the first means is disposed such that the blood enters the first means through the rough side; and
second means for transferring the plasma through the smooth side of the first means by virtue of capillarity from the second means, the second means having a first opening.

12. The combination of claim 11, wherein the first opening is disposed in proximity of the smooth side of the first means such that the first means is disposed between the second means and the blood, and wherein the second means transfers, by capillarity, plasma from the blood through the first opening into the interior of the second means.

13. The combination of claim 11, wherein the rough side of the first means is disposed for engaging the blood, the combination further comprising:
means for forming a cavity and carrying the first means, the cavity having an interior, such that the first means is disposed across the cavity and between the interior of the cavity and the blood,
wherein the first opening is disposed in proximity of the smooth side of the first means such that the first means is disposed between the second means and the blood, and wherein the second means, responsive to a period of time for the blood to engage passing, transfers, by capillarity, plasma from the blood through the smooth side of the first means and through the first opening into the interior of the second means.

14. The combination of claim 11 wherein the rough side of the first means is disposed for engaging the blood, the system further comprising:
means for collecting plasma, disposed below the first means, including an interior,
wherein the second means includes a second opening opposite the first opening, wherein the second means, responsive to a period of time for the blood to engage passing, and responsive to the first opening being disposed in proximity of the smooth side of the first means, transfers plasma from the blood through the smooth side of the first means and through the first opening of the second means into the interior of the second means by one or both of capillarity and/or gravity, and wherein the second means further transfers, by one or both of capillarity and/or gravity, plasma from the interior of the second means through the second opening of the second means into the interior of the means for collecting plasma.

15. The combination of claim 11, further comprising:
coating means for coating the interior of the second means;
wherein the second means transfers, by capillarity, the blood through the first opening into the interior of the second means, wherein, responsive to a period of time for the blood to mix with the coating means passing, and responsive to positioning the second means vertically, the second means transfers, by gravity and capillarity from the rough side of the first means, blood cells from the second means through the first opening of the second means to engage the rough side of the first means.
